(19) United States
(12) Patent Application Publication
(10) Pub. No.: US 2005/0113909 A1
(43) Pub. Date: May 26, 2005

(54) Polymer coated stents

(76) Inventors: Donald T. Shannon, Trabuco Canyon, CA (US); Chris Kuo, Anaheim, CA (US); Benny Tu, Lake Forest, CA (US)

Correspondence Address: EDWARDS LIFESCIENCES CORPORATION, ONE EDWARDS WAY, LEGAL DEPARTMENT, IRVINE, CA, 92614, US

(21) Appl. No.: 10/928,672

(22) Filed: Aug. 26, 2004

Related U.S. Application Data
(63) Continuation of application No. 10/001,765, filed on Oct. 31, 2001, now abandoned.
Application No. 10/001,765 is a continuation-in-part of application No. 09/358,350, filed on Jul. 21, 1999, now Pat. No. 6,790,225.
Application No. 09/358,350 is a division of application No. 08/675,644, filed on Jul. 3, 1996, now Pat. No. 5,928,279.

(51) Int. Cl. A61F 2/06

(52) U.S. Cl. 623/1.46; 623/1.15

(57) ABSTRACT

A polymer coated stent is disclosed. The stent may be a self-expanding stent or a balloon-expandable stent. The metal surface of the stent is coated with a polymer for enhanced biocompatibility. Amongst the various polymers that can comprise the coating of the stent are fluorine-containing polymers such as polytetrafluoroethylene (PTFE). Methods are also disclosed for depositing the coating on the surface of the stent.

This is a continuation of U.S. patent application Ser. No. 10/001,765, filed on Oct. 31, 2001, which is a continuation in part of application Ser. No. 09/358,350 filed Jul. 21, 1999, now pending, which is a division of U.S. patent application Ser. No. 08/675,644, filed Jul. 3, 1996, now issued as U.S. Pat. No. 5,928,279.

### BACKGROUND ART

The present invention pertains generally to medical devices and their methods of manufacture, and more particularly to tubular grafts having integral, radially expandable stents, for implantation in a cavities or passageways (e.g., ducts or blood vessels) of the body.

A. Stents

The prior art has included a number of radially expandable stents which may be initially deployed in a radially collapsed state suitable for transluminal insertion via a delivery catheter, and subsequently transitioned to a radially expanded state whereby the stent will contact and engage the surrounding wall or the anatomical duct or body cavity within which the stent has been positioned. Such stents have been used to support and maintain the patency of blood vessel lumens (e.g., as an adjuvant to balloon angioplasty) and to structurally support and/or anchor other apparatus, such as a tubular endovascular grafts, at desired locations within a body cavity or passageway (e.g., to anchor a tubular endovascular graft within a blood vessel such that the graft forms an internal conduit through an aneurysm or site of traumatic injury to the blood vessel wall).

Many stents of the prior art have been formed of individual member(s) such as wire, plastic, metal strips, or mesh that have been bent, woven, interlaced or otherwise fabricated into a generally cylindrical configuration. These stents of the prior art have generally been classified into two major categories: a) “self-expanding” stents, and b) “pressure expandable” stents. Some examples of stents of the prior art include those described in U.S. Pat. No. 5,405,377 (Cragg); U.S. Pat. No. 5,882,335 (Leone, et al.; U.S. Pat. No. 6,017,362 (Lau); U.S. Pat. No. 6,066,168 (Lau); U.S. Pat. No. 6,086,604 (Fischell et al.) and U.S. Pat. No. 6,117,165 (Becker).

i) Self-Expanding Stents

Self-expanding stents are typically formed of spring metal, shape memory alloy, or other material which is resiliently biased toward its fully radially expanded configuration or otherwise capable of self-expanding to its fully radially expanded configuration without the need for the exertion of outwardly directed radial force upon the stent by some extraneous expansion apparatus (e.g., a balloon or mechanical expander tool). These self-expanding stents may be initially radially compressed and loaded into a small diameter delivery catheter or alternatively mounted upon the outer surface of a delivery catheter equipped with some means for restraining or maintaining the stent in its radially compressed state. Thereafter, the delivery catheter is inserted into the body and is advanced to a position where the stent is located at or near the site at which it is to be implanted. Thereafter, the stent is expelled out of (or released from) the delivery catheter and allowed to self-expand to its full radial diameter. Such expansion of the stent causes the stent to frictionally engage the surrounding wall of the body cavity or passageway within which the stent has been positioned. The delivery catheter is then extracted, leaving the self-expanded stent at its intended site of implantation. Some examples of self-expanding stents of the prior art include those described in U.S. Pat. No. 4,655,771 (Wallsten et al.); U.S. Pat. No. 4,954,126 (Wallsten): U.S. Pat. No. 5,061,275 (Wallsten et al.); U.S. Pat. No. 4,580,568 (Gianturco); U.S. Pat. No. 4,830,003 (Wolf et al.); U.S. Pat. No. 5,035,706 (Gianturco et al.) and U.S. Pat. No. 5,330,400 (Song).

ii) Pressure-Expandable Stents

The pressure-expandable stents of the prior art are typically formed of metal wire, metal strips, or other malleable or plastically deformable material, fabricated into a generally cylindrical configuration. The pressure-expandable stent is initially disposed in a collapsed configuration having a diameter that is smaller than the desired final diameter of the stent, when implanted in the blood vessel. The collapsed stent is then loaded into or mounted upon a small diameter delivery catheter. The delivery catheter is then advanced to its desired location within the vasculature, and a balloon or other stent-expansion apparatus (which may be formed integrally of or incorporated into the delivery catheter) is utilized to exert outward radial force on the stent, thereby radially expanding and plastically deforming the stent to its intended operative diameter whereby the stent frictionally engages the surrounding blood vessel wall. The material of the stent undergoes plastic deformation during the pressure-expansion process. Such plastic deformation of the stent material causes the stent to remain in its radially expanded operative configuration. The balloon or other expansion apparatus is then deflated/collapsed and is withdrawn from the body separately from, or as part of, the delivery catheter, leaving the pressure-expanded stent at its intended site of implantation.

Some examples of pressure-expandable stents of the prior art include those described in U.S. Pat. No. 5,135,536 (Hillstead); U.S. Pat. No. 5,161,547 (Tower); U.S. Pat. No. 5,292,331 (Boneau); U.S. Pat. No. 5,304,200 (Spaulding) and U.S. Pat. No. 4,733,665 (Palmaz).

B. Elastomer Vascular Grafts

Elastomers, including fluoropolymers such as polytetrafluoroethylene, have been heretofore used for the manufacture of various types of prosthetic vascular grafts. These vascular grafts are typically of tubular configuration so as to be useable to replace an excised segment of blood vessel.

The tubular elastomer vascular grafts of the prior art have traditionally been implanted, by open surgical techniques, whereby a diseased or damaged segment of blood vessel is surgically excised and removed, and the tubular bioprosthetic graft is then anastomosed into the host blood vessel as a replacement for the previously removed segment thereof. Alternatively, such tubular prosthetic vascular grafts have also been used as bypass grafts wherein opposite ends of the graft are sutured to a host blood vessel so as to form a bypass conduit around a diseased, injured or occluded segment of the host vessel.

In general, many tubular prosthetic vascular grafts of the prior art have been formed of extruded, porous PTFE tubes. In some of the tubular grafts of the prior art, a PTFE tape is wrapped about and laminated to the outer surface of a tubular base graft to provide reinforcement and additional burst strength. Also, some of the prior tubular prosthetic vascular grafts have included external support member(s), such as a PTFE beading, bonded or laminated to the outer surface of the tubular graft to prevent the graft from becoming compressed or kinked during implantation. These externally supported tubular vascular grafts have proven to be particularly useful for replacing segments of blood vessel which pass through, or over, joints or other regions of the body which undergo frequent articulation or movement.

One commercially available, externally-supported, tubular vascular graft is formed of a PTFE tube having a PTFE filament helically wrapped around, and bonded to, the outer surface of the PTFE tube. (IMPRA Flex™ Graft, IMPRA, Inc., Tempe, Ariz.)

One other commercially available, externally-supported, tubular vascular graft comprises a regular walled, PTFE tube which has PTFE reinforcement tape helically wrapped around, and bonded to, the outer surface of the PTFE tube and individual rings of Fluorinated Ethylene Propylene (FEP) rings disposed around, and bonded to, the outer surface of the reinforcement tape. (FEP ringed ePTFE vascular graft, W.L. Gore & Associates, Inc., Flagstaff, Ariz.)

C. Stented Grafts

The prior art has also included a number of “stented grafts”. These stented grafts typically comprise a self-expanding or pressure-expandable stent that is affixed to or formed within a pliable tubular graft. Because of their radial compressibility/expandability, these stented grafts are particularly useable in applications wherein it is desired to insert the graft into an anatomical passageway (e.g., blood vessel) while the graft is in a radially compact state, and to subsequently expand and anchor the graft to the surrounding wall of the anatomical passageway. More recently, methods have been developed for introducing and implanting tubular prosthetic vascular grafts within the lumen of a blood vessel, by percutaneous or minimal incision means. Such endovascular implantation initially involves transluminal delivery of the graft, in a compacted state, by way of a catheter or other transluminally advancable delivery apparatus. Thereafter, the graft is radially expanded and anchored to the surrounding blood vessel wall, thereby holding the graft at its intended site of implantation within the host blood vessel. An affixation apparatus such as a stent may be utilized to anchor at least the opposite ends of the tubular graft to the surrounding blood vessel wall. One particular application for endovascular grafts of this type is in the treatment of vascular aneurysms without requiring open surgical access and resection of the aneurysmic blood vessel. Also, such stented grafts may also be useable to treat occlusive vascular disease—especially in cases where the stented graft is constructed in such a manner that the tubular graft material forms a complete barrier between the stent and the blood that is flowing through the blood vessel. In this manner the tubular graft material may serve as a smooth, biologically compatible, inner “covering” for the stent, thereby preventing a) turbulent blood-flow as the blood flows over the wire members or other structural material of which the stent is formed, b) immunologic reaction to the metal or other material of which the stent is formed, and c) a barrier to separate a diseased or damaged segment of blood vessel from the blood-flow passing therethrough. Such prevention of turbulent blood-flow and/or immunologic reaction to the stent material is believed to be desirable as both of these phenomena are believed to be associated with thrombus formation and/or restenosis of the blood vessel. Other uses for stented grafts may include restoring patency to, or re-canalizing, other anatomical passageways such as ducts of the biliary tract, digestive tract and/or genitourinary tract.

A number of specific desiderata are of special importance with regard to the suitability of particular expandable stent designs for incorporation into a stented graft. Among these are high flexibility, high hoop strength of the stent in its expanded form, minimal foreshortening of the stent in the course of its transition from a compressed state to an expanded state, and minimal “dog bone effect.” High flexibility is necessary in order for the stented graft to be smoothly inserted into regions of convolution. High hoop strength is necessary in order that the stent will fulfill its primary function of holding a lumen open. Minimal foreshortening is necessary to avoid excessive puckering, wrinkling or invagination of the elastomer graft material during expansion of the stent from its compressed state to its expanded state. The “dog bone effect” is the tendency of the ends of a stent to expand before the middle portion expands. This results in a “bone-shaped” structure in which the ends of the stent have expanded more than the middle portions. In addition to other undesirable characteristics of this expansion mode, excessive foreshortening accompanies “dog-boning.” Thus there remains a need for improved stented grafts having high flexibility, high hoop strength of the stent in its expanded form, minimal foreshortening of the stent in the course of its transition from a compressed state to an expanded state, and minimal “dog bone effect.” Variations on the known medical use of stented grafts have not been forthcoming, despite recent developments in the technology related to stent technology. Even though stented grafts are used extensively in medical practice, prior devices, products, or methods available to medical practitioners have not adequately addressed the need for advanced methods and apparatus for minimizing the deficiencies as set forth above.

The present invention embraces and finally addresses the clear need for advanced methods and apparatus for minimizing the deficiencies as set forth above. Thus, as pioneers and innovators attempt to make methods and apparatus for stented grafts cheaper, more universally used, and of higher quality, none has approached the desiderata outlined above in combination with simplicity and reliability of operation, until the teachings of the present invention. It is respectfully submitted that other references merely define the state of the art or show the type of systems that have been used to alternately address those issues ameliorated by the teachings of the present invention. Accordingly, further discussions of these references has been omitted at this time due to the fact that they are readily distinguishable from the instant teachings to one of skill in the art.

### OBJECTS AND SUMMARY OF THE INVENTION

It is an object of the present invention to provide a stented graft of high flexibility. It is another object of the present invention to provide a stented graft of high hoop strength of the stent in its expanded form. It is still another object of the present invention to provide a stented graft having minimal foreshortening of the stent in the course of its transition from a compressed state to an expanded state. It is yet still another object of the present invention to provide a stented graft having minimal “dog bone effect” in the course of its transition from a compressed state to an expanded state. It is even yet still another object of the present invention to provide a stented graft having minimal puckering, wrinkling or invagination of the elastomer graft material during expansion of the stent from its compressed state to its expanded state. It is a further object of the present invention to provide a stented graft that can be smoothly inserted into regions of convolution.

These and other objects are accomplished by the parts, constructions, arrangements, combinations and subcombinations comprising the present invention, the nature of which is set forth in the following general statement, and preferred embodiments of which—illustrative of the best modes in which applicant has contemplated applying the principles—are set forth in the following description and illustrated in the accompanying drawings, and are particularly and distinctly pointed out and set forth in the appended claims forming a part hereof.

The present invention is directed to improved tubular stented grafts and their methods of manufacture. The present invention may exist in numerous embodiments, including those wherein the stent component of the graft is formed integrally (i.e., within) the tubular graft, externally (i.e., on the outer surface of) the tubular graft, or internally (i.e., on the inner luminal surface) of the tubular graft. Embodiments of the invention may be self-expanding (i.e., incorporating a self-expanding stent) or pressure-expandable (i.e., incorporating a pressure-expandable stent).

In accordance with one embodiment of the invention, there is provided an improved integrally stented elastomer graft which comprises a tubular base graft, a radially expandable stent surrounding the outer surface of the tubular base graft, and an outer elastomer layer. The tubular outer layer is fused to the tubular base graft through lateral openings or perforations formed in the stent. A polymer coating, such as a PTFE coating, may be disposed on the stent to further facilitate fusing or boding of the stent to the base tube and/or outer tubular layer.

In accordance with another embodiment of the invention, there is provided an improved externally stented, tubular elastomer graft which comprises a radially compressible/expandable stent having a elastomer tube coaxially disposed within the stent, with the outer surface of the tubular elastomer graft being fused or attached to the stent. A polymer coating, such as PTFE or any other plastic that may be fused or adhered to PTFE, may be applied to or formed on the stent to facilitate the desired fusion or attachment of the tube graft to the stent, and/or to improve the biocompatibility of the stent.

In accordance with still another embodiment of the invention, there is provided an improved internally stented, tubular elastomer graft comprising a tubular outer layer and a radially expandable stent. The stent is coaxially disposed within the lumen of the tubular outer layer, and fused or attached thereto. The stent may be covered with a polymer coating, such as PTFE or other biocompatible plastic capable of adhering or fusing to PTFE, to facilitate the desired fusion or attachment of the stent to the outer tubular layer, and/or to improve the biocompatibility of the stent. Additionally or alternatively, PTFE particles may be disposed between the tubular outer layer and the tubular base graft to facilitate adhering or fusing of these two layers to one another, and/or to the stent. Such PTFE particles may be disposed between the inner base graft and outer tubular layer by applying or depositing PTFE liquid dispersion therebetween, or by depositing dry PTFE resin powder therebetween.

The invention may be manufactured by a method which comprises the steps of: a) initially positioning a generally cylindrical stent of either the self-expanding or pressure-expandable variety in contacting coaxial relation with the tubular base graft and/or the tubular outer layer, upon a cylindrical mandrel or other suitable support surface, and b) subsequently fusing the fuse (i.e., heating to a lamination temperature) assembled components (i.e., the stent in combination with the inner base graft and/or outer tubular layer) of the stented graft into a unitary stented graft structure. In integrally stented embodiments where both the tubular base graft and the tubular outer layer are present, such heating will additionally cause the tubular outer layer to fuse to the inner tubular base graft, through lateral openings or perforations which exist in the stent. In instances where a plastic coating is formed on the stent, such coating may be in the nature of a tube or film that is applied to the stent prior to assembly and mounting of the stented graft components on the mandrel or other support surface.

By the above-described materials and methods of construction, the stented elastomer grafts of the present invention are capable of radially expanding and contracting without excessive puckering, wrinkling or invagination of the graft material. Furthermore, in embodiments wherein the stent is constructed of individual members which move or reposition relative to one another during respective expansion and contraction of the stented graft, the manufacturing methods and materials of the present invention render the elastomer sufficiently strong and sufficiently firmly laminated or fused so as to permit such relative movement of the individual members of the stent without tearing or rupturing of the tubular graft.

Further objects and advantages of the invention will become apparent to those skilled in the art upon reading and understanding the following detailed description and the accompanying drawings.

### BRIEF EXPLANATION OF THE DRAWINGS

FIG. 1 is a perspective view of an integrally stented tubular graft of the present invention, wherein a portion of the graft has been inserted into a tubular catheter.

FIG. 1 a is an enlarged perspective view of a segment of FIG. 1.

FIG. 2 is an enlarged, cut-away, elevational view of a preferred, integrally stented, tubular graft of the present invention.

FIG. 3a is an enlarged perspective view of a portion of the stent incorporated in the graft of FIG. 2.

FIG. 3b is an enlarged cross-sectional view through line 3b-3d of FIG. 3a.

FIGS. 4a-4f are a step-by-step illustration of a preferred method for manufacturing an integrally stented PTFE graft of the present invention.

FIG. 5 is a schematic illustration of an alternative electron beam deposition method which is usable for depositing PTFE coating on the stent portion of the integrally stented PTFE grafts of the present invention.

FIG. 6 is a perspective view of an alternative heating apparatus which is useable in the manufacture of the integrally stented PTFE grafts of the present invention.

FIG. 7 is a perspective view of a section of a stent of the invention that illustrates portions of three elements each comprising an undulating zigzag shape.

FIG. 7a is an enlarged longitudinal sectional view of a stent of the invention shown in FIG. 7 taken along section line 7a therein.

FIG. 8 is a perspective view of a section of a stent of the invention that illustrates portions of two elements each comprising an undulating sinusoidal shape.

FIG. 8a is an enlarged longitudinal sectional view of a stent of the invention shown in FIG. 8 taken along section line 8a therein.

### DETAILED DESCRIPTION OF THE PREFERRED EMBODIMENTS

The following detailed description is provided for the purpose of describing and illustrating presently preferred embodiments of the invention only, and is not intended to exhaustively describe all possible embodiments in which the invention may be practiced.

A. The Structure of an Integrally Stented PTFE Graft

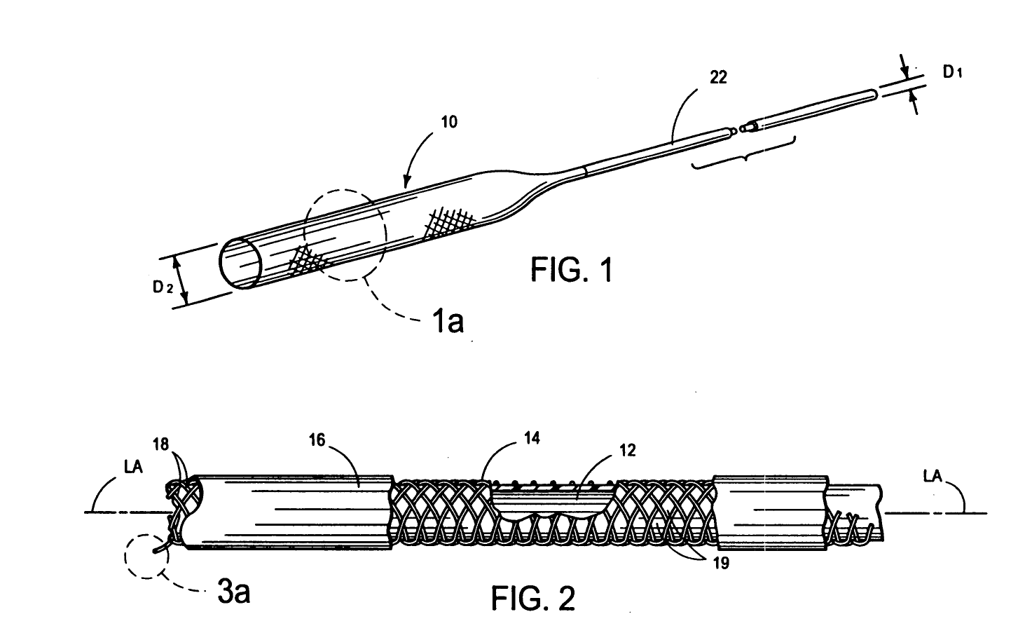

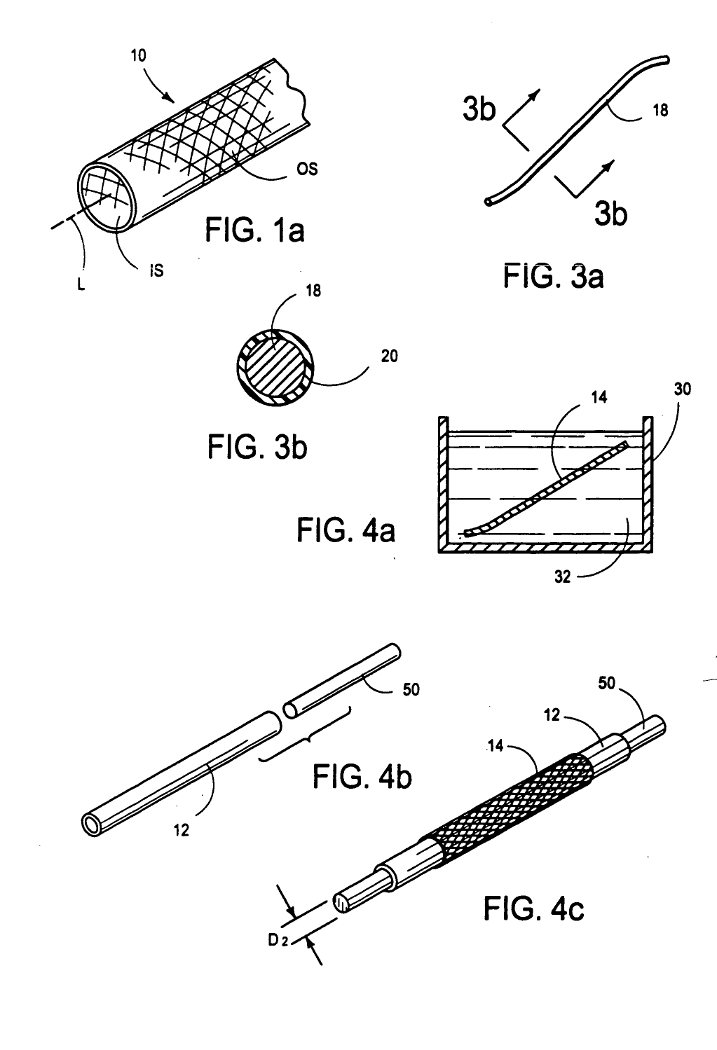

With reference to FIGS. 1-3b, there is shown an integrally stented tubular graft 10 of the present invention. The preferred integrally stented graft 10 comprises a tubular base graft 12, a PTFE-coated stent 14 and an outer layer of elastomer 16. Stent 14 is formed of metal, such as an alloy of cobalt, chromium, nickel or molybdenum, wherein the alloying residue is iron. One specific example of a commercially available alloy which may is usable to form the wires 18 of the stent 14 is Elgiloy (The Elgiloy Company, 1565 Fleetwood Drive, Elgin, Ill. 60120. Stent 14 may be radially compressed to a smaller diameter D₁ and radial constraint, as may be applied by the surrounding wall of the tubular delivery catheter 22 shown in FIG. 1, may be applied to hold the stent 14 in such radially compressed state (diameter D₁). Thereafter, when the radial constraint is removed from the stent 14, the stent 14 will resiliently spring back to its radially expanded diameter D₂.

B. Preparation of the PTFE Tubular Base Graft

i.) Preparation of Paste

The manufacture of the tubular base graft begins with the step of preparing a PTFE paste dispersion for subsequent extrusion. This PTFE paste dispersion may be prepared by known methodology whereby a fine, virgin PTFE powder (e.g., F-104 or F-103 Virgin PTFE Fine Powder, Dakin America, 20 Olympic Drive, Orangebury, N.Y. 10962) is blended with a liquid lubricant, such as odorless mineral spirits (e.g., Isopar®, Exxon Chemical Company, Houston, Tex. 77253-3272), to form a PTFE paste of the desired consistency.

ii.) Extrusion of Tube

The PTFE-lubricant blend dispersion is subsequently passed through a tubular extrusion dye to form a tubular extrudate.

iii.) Drying

The wet tubular extrudate is then subjected to a drying step whereby the liquid lubricant is removed. This drying step may be accomplished at room temperature or by placing the wet tubular extrudate in an oven maintained at an elevated temperature at or near the lubricant's dry point for a sufficient period of time to result in evaporation of substantially all of the liquid lubricant.

iv.) Expansion

Thereafter, the dried tubular extrudate is longitudinally expanded or longitudinally drawn at a temperature less than 327° C. and typically in the range of 250-326° C. This longitudinal expansion of the extrudate may be accomplished through the use of known methodology, and may be implemented by the use of a device known as a batch expander. Typically, the tubular extrudate is longitudinally expanded by an expansion ratio of more than two to one (2:1) (i.e., at least two (2) times its original length).

v.) Sintering

After the longitudinal expansion step has been completed, the expanded PTFE tube is subjected to a sintering step whereby it is heated to a temperature above the sintering temperature of PTFE (i.e., 350-370° C.) to effect amorphous-locking of the PTFE polymer. The methodology used to effect the sintering step, and the devices used to implement such methodology, are known in the art. The PTFE tape 16 may be manufactured by any suitable method, including the general method for manufacturing expanded PTFE tape.

C. Coating of the Stent and/or Deposition of PTFE Between Layers to Enhance Bonding

Prior to assembly of the components of the integrally stented graft 10, the stent 14 may be coated with a polymer coating 20. The polymer coating formed on the stent 14 may be any suitable type of polymer that will adhere to PTFE. Examples of polymers that may be used for such polymer coating or covering include polytetrafluoroethylene (PTFE), fluorinated ethylene propylene (FEP), polytetrafluoroethylene-perfluoroalkyl vinyl ether copolymer (PFA), polyvinyl chloride (PVC), polypropylene (PP), polyethylene terephthalate (PET) polyvinylidene fluoride (PVDF) and other biocompatible plastics.

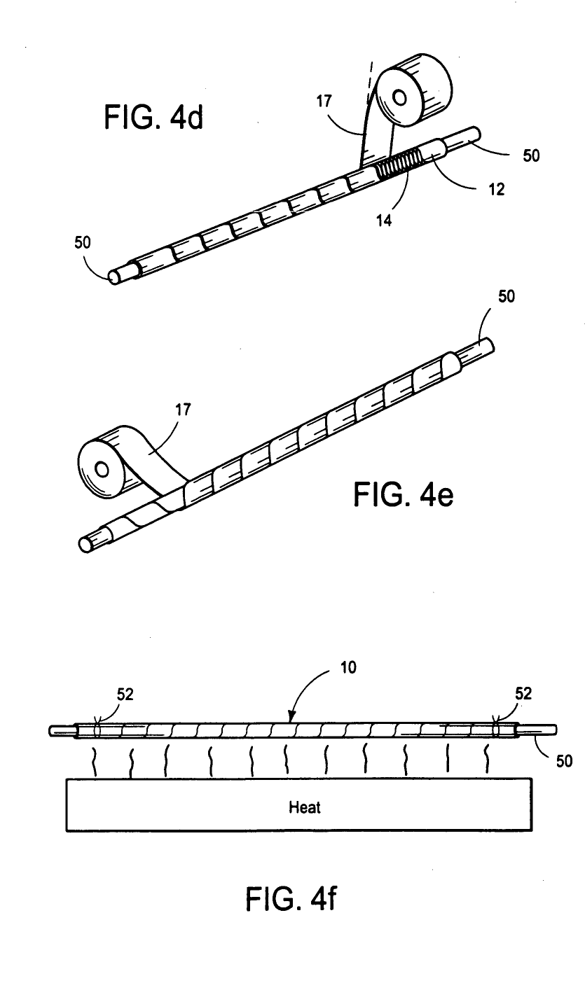

One manner in which such coating of the stent 14 may be carried out is illustrated in FIG. 4a. As shown in FIG. 4a, the stent 14 may be immersed in a vessel 30 containing an aqueous dispersion of PTFE 32. One aqueous PTFE dispersion which may be useable for coating of the stent 14 is DuPont T-30 Aqueous PTFE Dispersion, available commercially from the E.I. DuPont de Nemours Co., (Wilmington, Del.). Another commercially available PTFE dispersion 32 which may be utilized for coating of the stent is Daikin-Polyflon TFE Dispersion, available from Daikin Industries, Ltd., Chemical Division (Umeda Center Bldg., 4-12 chome, Nakazaki-nishi, Kita-ku, Osaka, Japan).

The time in which the stent 14 must remain immersed in the liquid PTFE dispersion 32 may vary, depending on the construction of the stent 14 and the chemical composition of the PTFE dispersion 32. However, in most cases, an immersion time of 10-15 seconds will be sufficient to obtain uniform deposition of the PTFE coating 20 on the wire members 18 of the stent 14. After the stent 14 has been removed from the liquid PTFE dispersion 32, it will be permitted to air dry such that a dry PTFE coating 20 remains deposited upon the outer surface of each wire 18 of the stent 14.

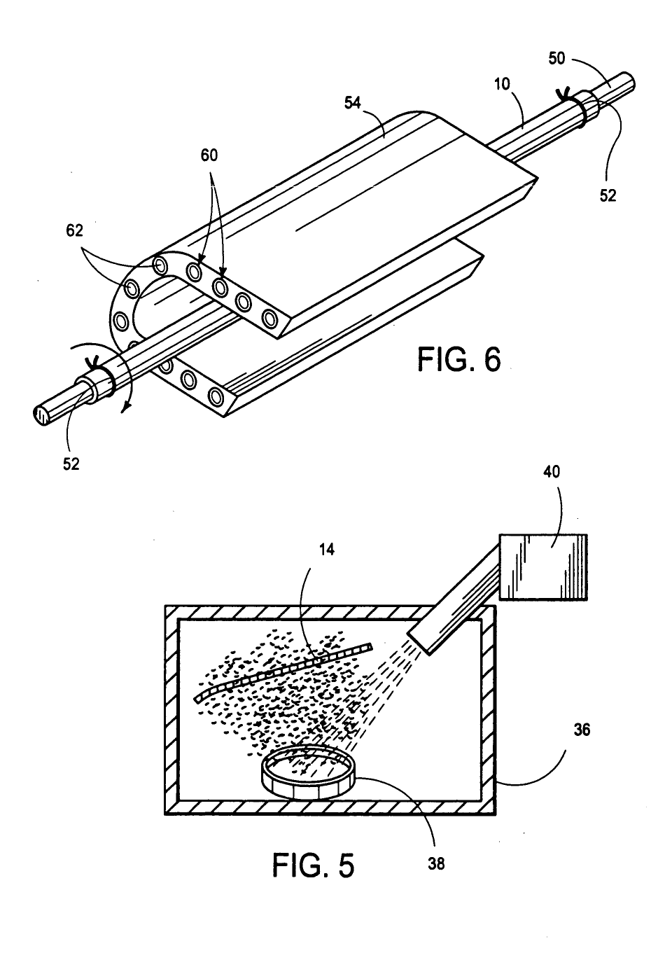

Optionally, after the air drying has been completed, the PTFE coated stent 14 may be placed in an oven at 350° C. for approximately 10 minutes to sinter the PTFE coating and/or to enhance the bonding of the PTFE coating 20 to wire members 18 of the stent 14. Sintering of the PTFE coating renders the coating more resistant to abrasion or peeling during the subsequent handing of the stent and/or the ensuing manufacture and use of the stented graft 10. It will be appreciated that various alternative methods, other than immersion, may be used for depositing the PTFE coating 20 on the stent 14. One alternative method is electron beam deposition, as illustrated in FIG. 5. In accordance with this alternative PTFE deposition method, the stent 14 is positioned within a closed vacuum chamber 36 wherein a mass of PTFE 38 is located. An electron beam apparatus 40 is then utilized to project electron beam radiation onto the PTFE 38 within the chamber 36 so as to cause sublimation of the PTFE and resultant deposition of the layer 20 of PTFE on the outer surface of the stent 14. The apparatus and specific methodology useable to perform this electron beam deposition of the PTFE coating 20 are well known to those of skill in the relevant art.

As with the above-described immersion process (FIG. 4a), the stent 14 whereupon the PTFE coating 20 has been deposited may be subjected to optional heating at 350° C. for a period of approximately ten minutes in order to sinter the PTFE coating and/or to enhance the bonding of the PTFE coating 20 to the wire members 18 of the stent 14. As an alternative to coating of the stent, or in addition thereto, such PTFE aqueous dispersion may be painted onto the outer surface of the base graft 12, or the inner surface of the outer tubular layer 16, or may be otherwise disposed between the base graft 12 and outer tubular layer 16 to facilitate fusion or bonding of the inner base graft 12 to the outer tubular layer 16. Or, such PTFE aqueous dispersion may be sprayed or otherwise applied to the outer surface of the outer tubular layer 16 provided that the PTFE present in the dispersion are small enough to migrate inwardly through pores in the outer tubular layer 16, thereby becoming deposited between the outer tubular layer 16 and the inner base graft 12.

Another alternative or additional means by which adherence or fusion of the base graft 12, outer tubular layer 16 and/or stent 14 may be facilitated or enhanced includes the deposition of raw PTFE resin powder between the outer tubular layer 16 and inner base graft 12, and/or upon the stent 14. It will be appreciated that in many cases, it will be desirable to apply the polymer coating 20 to the stent 14 while the stent 14 is in its fully radially expanded configuration of diameter D₂. In this manner, after the coating 20 has been applied and formed on the fully radially expanded stent 14, the stent 14 may subsequently be contracted to its radially compact configuration of diameter D₁ without tearing or disrupting of the previously-applied coating 20. In embodiments that utilize a pressure-expandable stent 14, it may thus be necessary to volitionally or purposely expand the stent 14 to its fully radially expanded diameter D₂ prior to application of the coating 20. Alternatively, when the stent 14 is of the self-expanding variety it will, in most cases, automatically assume its fully radially expanded configuration of diameter D₂ and no such volitional or purposeful pre-expansion of the stent 14 will be required.

D. Assembly and Construction of the Integrally Stented PTFE Graft

FIGS. 4b-4f show, in step-wise fashion, the preferred method for assembling and constructing the integrally stented PTFE graft 10.

As shown in FIG. 4b, the tubular base graft 12 is initially disposed on a rod or mandrel 50. Such rod or mandrel 50 may comprise a stainless steel rod having an outer diameter which is only slightly smaller than the inner diameter of the tubular base graft 12. In this manner, the tubular base graft 12 may be slidably advanced onto the outer surface of the mandrel 50 without undue effort or damage to the base graft 12.

Thereafter, the PTFE-coated stent 14 is axially advanced onto the outer surface of the tubular base graft 12, as shown in FIG. 4c. At this point in the process, PTFE liquid dispersion or powdered PTFE resin may be additionally (optionally) applied to the stent 14 and/or outer surface of the base graft 12 to promote further bonding and fusion of the base graft 12 to the stent 14 and/or subsequently applied outer layer 16. In this regard, the mandrel-borne tubular base graft 12 and stent 14 may be rolled in powdered PTFE resin to accomplish the desired deposition of PTFE powder thereon. Alternatively, the above-described PTFE liquid dispersion may be painted sprayed or otherwise applied to the surface of the stent 14 and/or outer surface of the tubular base graft 12 prior to subsequent application of the outer tubular layer 16.

Thereafter, as shown in FIG. 4d, the tape 17 is initially helically wrapped in overlapping fashion, on the outer surface of the stent 14, in a first direction. In the preferred embodiment, tape of ½ inch width is used. The tape is helically wrapped about the stent at a pitch angle whereby 6 to 8 revolutions of the tape are applied per linear inch of the stent 14. Thereafter, as shown in FIG. 4e, a second tape wrap in the opposite direction is accomplished, preferably using the same width of tape at the same pitch angle, thereby applying another 6-8 revolutions of tape 17 per linear inch of stent 14. In this manner, both wrappings of the tape 17 (FIGS. 4d and 4e) combine to form a tubular, outer PTFE layer 16 which preferably has a thickness of less than 0.1 inches, and which may be formed of 1 to 10 consecutive (e.g., laminated) layers of the tape 17, for example, when using ePTFE tape of less than 1.6 g/cc density and ½ inch width, the first helical wrap (FIG. 4d) may deposit four consecutive layers of tape 17 and the second helical wrap (FIG. 4e) may deposit an additional 4 layers of tape 17, thereby resulting in an outer tubular layer 16 which is made up of a total of 8 layers of such tape 17.

Optionally, to further promote bonding of the outer tubular layer 16 to the stent 14 and/or inner base graft 12, liquid PTFE dispersion may be sprayed, painted or otherwise applied to and dried upon the tape 17 prior to wrapping, or such liquid PTFE dispersion may be deposited by any suitable means (spraying, painting, etc.) between the outer tubular layer 16 formed by the helically wrapped tape 17 and the inner base graft 12. Or such liquid PTFE dispersion may be sprayed onto or otherwise applied to the outer surface of the helically wrapped tape 17 such the small particles of PTFE contained within the liquid dispersion will migrate inwardly through pores in the layers of tape 17, and will thereby become deposited between the outer tubular layer 16 and the inner base graft 12 prior to subsequent heating of the assembly, as described herebelow. Another alternative (and optional) method for depositing polymer (e.g., PTFE) particles between the base graft 12 and outer tubular layer 16 is by rolling the mandrel 50, having the base graft 12 and stent 14 disposed thereon, in dry, powdered polymer resin (e.g., the above-described PTFE resin) to cause such dry polymer resin to become deposited on the outer surface of the base graft 12 and/or stent 14 prior to application of the tape 17 as shown in FIGS. 4d and 4e.

Thereafter, as shown in FIG. 4f, ligatures 52 of stainless steel wire are tied about the opposite ends of the graft 10 so as to securely hold the base graft 12, PTFE-coated stent 14 and outer layer 16 on the mandrel 50. The mandrel, having the graft 10 disposed thereon is then heated to a temperature of 363° C.±2° C. for thirty minutes. Such heating will cause the outer PTFE layer 16 to heat fuse to the inner base graft 12 through the openings 19 which exist in the stent 14, and will further facilitate bonding or fusing of the PTFE coating 20 of the stent 14 to the adjacent base graft 12 and outer tape layer 16. In this manner, the desired integrally-stented PTFE tubular graft 10 is formed.

The heating step illustrated schematically in FIG. 4f may be carried out by any suitable means. For example, the mandrel 50 having the graft 10 and ligatures 52 disposed thereon may be placed in an oven preheated to the desired temperature, for the desired period of time. Alternatively, the mandrel, having the graft 10 and ligatures 52 disposed thereon may be rolled on a hot plate or heated surface to accomplish the desired heat fusing or bonding of the outer layer 16, base graft 12 and PTFE coating 20 of the stent 14.

Another alternative apparatus that may be utilized for the heating step shown schematically in FIG. 4f, is the U-shaped aluminum block heater shown in FIG. 6. This aluminum block heater is formed of a solid aluminum plate or block 54 formed into a generally U-shaped configuration, and having a plurality of bore holes 60 formed longitudinally therein and extending at least part way therethrough. Elongate, cylindrical, electric heaters 62, such as those commercially available from the Watlow Electric Company, 12001 Lackland Road, St. Louis, Mo. 63146, are inserted into the bore holes 60, and such heaters 62 are heated to a temperature which will cause the inner surface of the U-shaped aluminum heater block 54 to be maintained at approximately 300° C. or greater. It will be appreciated that other types of heating apparatus, such as electrical strip heaters mounted on the outer surface of the U-shaped block 54, may be useable as an alternative to the bore holes 60 and cylindrical heaters 62 described herein.

After the U-shaped block 54 has been heated to the desired temperature, the mandrel 50, having the graft 10 and ligatures 52 disposed thereon, is inserted into the U-shaped inner region of the block 54, and is rotated, therein so as to accomplish the desired heat fusing of the tubular base graft 12, outer tape layer 16 and PTFE coating 20 of the stent 14.

E. Assembly and Construction of Internally Stented Tube Graft

In one embodiment of the invention, the inner base graft 12 may be eliminated or excluded, thereby providing a modified version of the stented graft 10 comprising only the stent 14 and outer tubular layer 16. Here, the above-described manufacturing method is performed as described without the tubular base graft 12, thereby forming a modified version of the stented graft 10 wherein the outer tubular layer 16 is fused only to the stent 14.

In embodiments wherein the stent 14 is coated with a polymer coating such as PTFE, the presence of such coating on the stent 14 will provide lubricity and biocompatibility, which may render such internally stented graft suitable for use in applications wherein the exposed stent 14 will come in direct contact with biological fluid or blood flowing through the graft, thereby avoiding the need for use of the internal base graft 12. Thus, this embodiment of the present invention includes all possible embodiments wherein only the outer tubular layer 16 is utilized in conjunction with the stent 14, to provide an internally stented graft 10 which is devoid of any internal tubular base graft 12.

F. Assembly and Construction of Externally Stented Tube Graft

In an alternative embodiment of the invention, the outer tubular layer 16 may be excluded or eliminated, thereby providing an externally stented tube graft which comprises only the stent 14 and the inner-base tube 12. Here, the above-described manufacturing method is performed as described without the outer tubular layer 16. This results in the formation of a modified version of the stented graft 10, comprising only the inner base graft 12 and the stent 14. In embodiments wherein the stent 14 is coated with a polymer coating, such as PTFE, the presence of such coating on the stent 14 will provide for enhanced biocompatability, which may render such externally stented graft suitable for implantation in blood vessels or other tubular anatomical passageways wherein the exposed exterior of the coated stent 14 comes in direct contact with vascular tissue or other tissue of the body, thereby avoiding the need for use of the outer tubular layer 16. Thus, this embodiment of the present invention includes all possible embodiments wherein only the inner base graft 12 is utilized in conjunction with the stent 14, to provide an externally stented graft 10 which is devoid of any outer tubular layer 16.

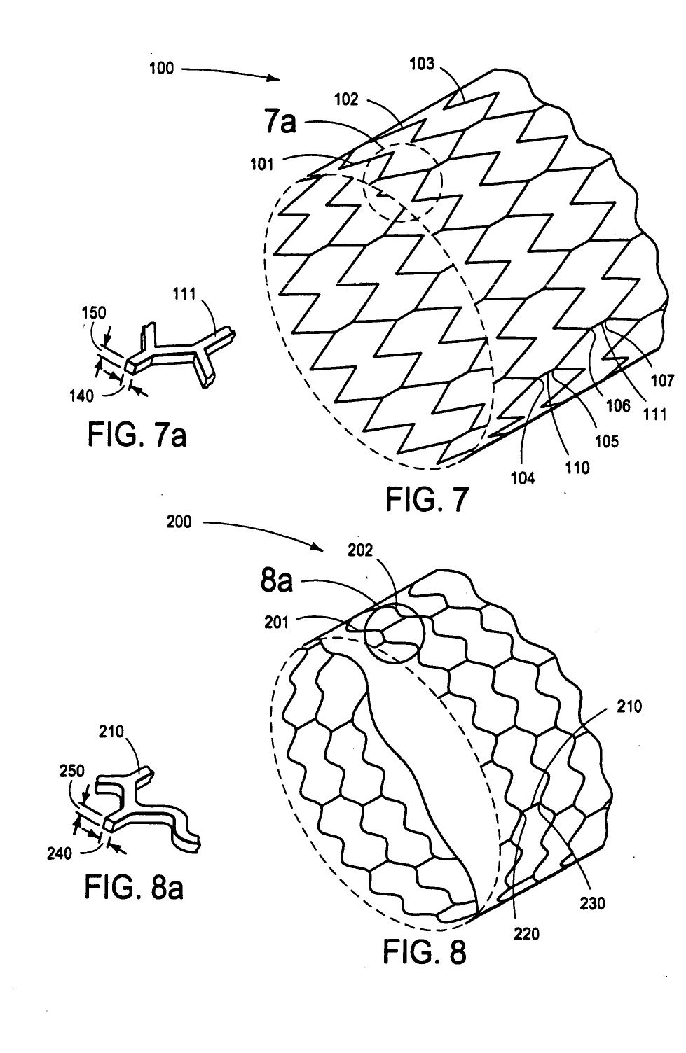

Referring now to FIG. 7 and FIG. 8, there are shown portions of two embodiments of the stent of the invention. They comprise a plurality of elements, wherein each element comprises an undulating shape formed into a generally cylindrical configuration having a cylinder axis, wherein each element is connected to an adjacent neighbor element by at least one linear connector. In FIG. 7, a portion of one embodiment of the stent is shown generally at 100. Stent portion 100 consists of three elements 101, 102 and 103, each of which comprises a zigzag pattern comprising a plurality of zigs having tips and a plurality of zags having tips. A tip 104 on a zig of element 101 and a nearest tip 105 of a zag of an adjacent neighbor element 105 generally lie in a plane passing through the cylinder axis, and are connected by a linear connector 105. Likewise, a tip 106 on a zig of element 102 and a nearest tip 107 on a zag of an adjacent neighbor element 103 generally lie in a plane passing through the cylinder axis, and are connected by a linear connector 111. Connector 111 is substantially circumferentially offset from adjacent neighbor connector 105. Stent 100 is constructed of material that has a width dimension 140 and a depth dimension 150 each of which is smaller than the length dimension of linear connectors 110 and 111. In FIG. 8, a portion of another embodiment of the stent is shown generally at 200. Stent portion 200 consists of two elements 201 and 202, each of which comprises an undulating pattern comprising a plurality of peaks and valleys. A valley 220 on element 201 and a nearest peak 230 of adjacent neighbor element 202 generally lie in a plane passing through the cylinder axis, and are connected by a linear connector 210. Stent 200 is constructed of material that has a width dimension 240 and a depth dimension 250 each of which is smaller than the length dimension of connector 210.

Thus, the invention comprises an improved stented graft that can alternately include a compact configuration having a first diameter and an expanded configuration having a greater diameter, comprising at least one stent formed in a generally cylindrical shape having an outer surface and a hollow bore extending longitudinally therethrough. The stent can alternately exist in a compact configuration having a first diameter, and an expanded configuration having a greater diameter and a plurality of lateral openings. In one embodiment, a flexible, porous, biocompatible tubular elastomer covering has a first end, a second end, an outer surface and a hollow bore that extends longitudinally therethrough to define an inner surface. The stent is deployed coaxially within the hollow bore of the covering such that the inner surface of the tubular covering is in contact with the outer surface of the stent. In another embodiment, a tubular inner graft formed of an elastomer has an outer surface and an inner surface, and is deployed coaxially within the hollow bore of the stent; whereby the outer surface of the tubular inner graft is in contact with the inner surface of the stent. Still another embodiment comprises a continuous, tubular PTFE covering formed on the stent. The PTFE covering comprises a tubular inner base graft formed of expanded, sintered PTFE, and has an outer surface and an inner surface. The tubular base graft is deployed coaxially within the hollow bore of the stent such that the outer surface of the tubular base graft is in contact with the inner surface of the stent, and the inner surface of the tubular base graft defines a luminal passageway through the stented graft. A tubular outer layer is formed of expanded, sintered PTFE tape that has a width of less than about 1 inch. The tape is wound about the outer surface of the stent to create the tubular outer layer, and the stent is captured between the outer layer and the tubular base graft. The tubular outer layer is attached to the tubular base graft through lateral openings in the stent to form an integrally stented, continuous PTFE tube which is alternately disposable in a radially compact configuration and a radially expanded configuration.

In the improvement of the invention, the stent comprises a plurality of elements. Each element comprises an undulating linear shape formed into a generally cylindrical configuration having a cylinder axis generally aligned on the axis of the hollow bore, and each element is connected to an adjacent neighbor element by at least one linear connector. The elements may comprise a spiral. One connector may be substantially circumferentially offset from an adjacent neighbor connector, and may form a helical array. Alternatively, a connector may not be substantially circumferentially offset from an adjacent neighbor connector. The undulating linear shape may be a generally zigzag shape comprising a plurality of zigs having tips and a plurality of zags having tips, wherein the tip of each zig of each element and the nearest the tip of each zig of an adjacent neighbor element generally lie in a plane passing through the axis of the hollow bore, and wherein the tip of at least one zig of each element and at least one nearest tip of a zig of an adjacent neighbor are connected by one linear connector.

Alternatively, the undulating linear shape may be a sinusoidal shape having a plurality of peaks and a plurality of valleys. Each peak of each element and each valley of an adjacent neighbor may lie generally in a common plane passing through the axis of the hollow bore, and at least one peak of each element and the valley of an adjacent neighbor lying generally in the common plane may be connected by one linear connector. The length of each linear connector is greater than its width or depth, and may be 3-10 times greater than the width or depth. The stent and elastomer may be anchored to each other by means for anchoring, such as protrusions of the covering that fixedly protrude into the lateral openings in the stent. The elastomer may be polytetrafluoroethylene, fluorinated ethylene propylene, polytetrafluoroethylene-perfluoroalkyl vinyl ether copolymer, polyvinyl chloride, polypropylene, polyethylene terephthalate, broad fluoride, other biocompatible plastics, and expanded, sintered PTFE (which may be tape) having fibrils measuring about 300μ-5μ in length. The tape may have a width of less than about 0.5 inches to about 1 inch, a thickness of less than 0.015 inch (0.038 cm.), and a density of less than 1.6 g/cc. The tape may be wound about the stent in overlapping fashion, for example, helically. The tape may be wound in a first direction and then in the opposite direction, and comprise 1 to 10 layers. The tape may be helically wrapped such that 6-8 revolutions of tape are applied per longitudinal inch (2.54 cm.) of the stented graft. The thickness of the covering may be less than 0.1 inch (0.25 cm.) The stented graft may include a self-expanding stent comprising a shape memory alloy that can alternately exist in a first and a second crystalline state, or it may include a pressure-expandable stent. The stent may be formed of a metal alloy comprising at least two elements selected from the group consisting of iron, cobalt, chromium, nickel, titanium, niobium, and molybdenum. For example, the alloy may comprise at least about 51% to about 59% nickel and the remainder comprising titanium. Alternatively, it may comprise about 0.25% chromium, at least about 51% to about 59% nickel, and the remainder comprising titanium.

In the improvement of the invention, the stent comprises a plurality of elements. Each element comprises an undulating linear shape formed into a generally cylindrical configuration having a cylinder axis generally aligned on the axis of the hollow bore, and each element is connected to an adjacent neighbor element by at least one linear connector. This improved stented graft of the invention thus includes a stent comprising a substantially continuous construction cut from tubing of an alloy listed above. The cutting process may involve laser cutting, electromechanical discharge, electrochemical etching, or a similar effective process known in the art. The cutting process is advantageously followed by a known deburring treatment to remove potential sharp areas.

The stent may further possess a polymer coating, such as polytetrafluoroethylene, fluorinated ethylene propylene, polytetrafluoroethylene-perfluoroalkyl vinyl ether copolymer, polyvinyl chloride, polypropylene, polyethylene terephthalate, polyvinylidene fluoride, and, other biocompatible plastics. The coating may be applied by immersing the stent in liquid polymer dispersion, removing the stent from the liquid polymer dispersion, and, drying the liquid polymer dispersion that has remained on the stent. Alternatively, the polymer coating is formed by electron beam deposition. The tubular covering may be adherent to the polymer coating.

A method for the treatment of cardiovascular disease comprises implanting the stented graft in a patient in need of such treatment wherein the implantation is effective to ameliorate one or more of the symptoms of the cardiovascular disease.

An article of manufacture comprises packaging material and the stented graft contained within the packaging material, wherein the stented graft is effective for implantation in a patient afflicted with cardiovascular disease, and the packaging material includes a label that indicates that the device is effective for the implantation.

It will be appreciated that the invention has been described hereabove with reference to certain presently preferred embodiments of the invention. Various additions, deletions, alterations and modifications may be made to the above-described embodiments without departing from the intended spirit and scope of the invention. For example, the linear connectors may collectively form arrays that may be helical, linear, or neither helical nor linear. Likewise, linear connectors may connect peaks to peaks, valleys to valleys, or peaks to valleys. Again, linear connectors may connect zigs to zigs, zags to zags, or zigs to zags. Accordingly, it is intended that all such reasonable additions, deletions, modifications and alterations to the above described embodiments be included within the scope of the following claims.

On this basis, the instant invention should be recognized as constituting progress in science and the useful arts, as solving the problems in cardiology enumerated above. In the foregoing description, certain terms have been used for brevity, clearness and understanding, but no unnecessary limitation are to be implied therefrom beyond the requirements of the prior art, because such words are used for descriptive purposes herein and are intended to be broadly construed.

Having described preferred embodiments of the invention with reference to the accompanying drawings, it is to be understood that the invention is not limited to those precise embodiments, and that the various changes and modifications may be effected therein by one skilled in the art without departing from the scope or spirit of the inventions defined in the appended claims. For example, the product can have other shapes, or could make use of other metals and plastics. Thus, the scope of the invention should be determined by the appended claims and their legal equivalents, rather than by the examples given. All changes that come within the meaning and range of equivalency of the claims are to be embraced within their scope.

### Definitions

Unless defined otherwise, all technical and scientific terms used herein have the same meaning as is commonly understood by one of skill in the art to which this invention belongs. All patents and publications referred to herein are incorporated in their entirety by reference.

1. An implantable medical device comprising:
a radially expandable stent comprising a generally cylindrical wall surface made of a metal, wherein the generally cylindrical wall surface comprises a plurality of lateral openings; and
a polymer coating on the surface of the metal.

2. The device of claim 1, wherein the stent comprises a plurality of undulating elements that comprise a spiral with a plurality of turns.

3. The device of claim 2, wherein the adjacent turns of the spiral are connected to each other by at least one linear connector.

4. The device of claim 2, wherein the undulating elements are zigzag elements.

5. The device of claim 2, wherein the undulating elements are sinusoidal elements.

6. The device of claim 1, wherein the polymer coating comprises fluorine.

7. The device of claim 6, wherein the polymer coating is PTFE

8. The device of claim 1, wherein the stent comprises a self-expanding stent.

9. The device of claim 8, wherein the self-expanding stent comprises a shape memory alloy.

10. The device of claim 1, wherein the stent comprises a balloon expandable stent.

11. An implantable device that has a compressed undeployed diameter and an expanded deployed diameter, the device comprising:
a radially expandable stent comprising a metal mesh; and
a polymer coating deposited on the surface of the metal mesh.

12. The device of claim 11, wherein the stent comprises a plurality of undulating elements that comprise a spiral with a plurality of turns.

13. The device of claim 12, wherein the adjacent turns of the spiral are connected to each other by at least one linear connector.

14. The device of claim 11, wherein the polymer coating comprises a fluorine compound.

15. The device of claim 14, wherein the fluorine compound is PTFE.

16. A method of manufacturing a PTFE coated stent, the method comprising:
(a) providing a stent comprising a metal mesh; and
(b) depositing a PTFE coating on the metal mesh.

17. The method of claim 16, wherein step (b) comprises immersing the stent in an aqueous dispersion of PTFE.

18. The method of claim 16, wherein step (b) comprises depositing PTFE on the metal mesh using electron beam deposition.

19. The method of claim 16, wherein the stent comprises a self-expanding stent.

20. The method of claim 16, wherein the stent comprises a balloon expandable stent.
